(19) United States
(12) Patent Application Publication
(10) Pub. No.: US 2024/0285507 A1
(43) Pub. Date: Aug. 29, 2024

(54) COSMETIC COMPOSITION COMPRISING AT LEAST ONE ANIONIC SURFACTANT, ONE AMPHOTERIC OR ZWITTERIONIC SURFACTANT, ONE OIL-IN-WATER EMULSION AND ONE PARTICULAR AGENT

(71) Applicant: L'OREAL, Paris (FR)

(72) Inventors: Saber MALOUG, SAINT-OUEN (FR); Anne-Laure FAMEAU, SAINT-OUEN (FR); Christian TU, SAINT-OUEN (FR)

(21) Appl. No.: 18/290,065

(22) PCT Filed: May 12, 2022
(86) PCT No.: PCT/EP2022/062977

§ 371 Date: Nov. 9, 2023

(30) Foreign Application Priority Data
May 12, 2021 (FR) FR2105047

(51) Int. Cl. A61K 8/898; A61K 8/06; A61K 8/44; A61K 8/46; A61Q 5/02

(52) U.S. Cl. CPC A61K 8/898; A61K 8/062; A61K 8/44; A61K 8/463; A61Q 5/02

(57) ABSTRACT

The present invention relates to a cosmetic composition comprising: a) at least one anionic surfactant; b) at least one amphoteric or zwitterionic surfactant; c) at least one oil-in-water emulsion having a particle size D50 of less than 350 nm, the size being expressed on a volume basis, and comprising: —a silicone mixture comprising (i) one or more polydialkylsiloxanes bearing trialkylsilyl end groups, having a viscosity at 25° C. ranging from 40000 to 100000 mPa·s and (ii) one or more amino silicones having a viscosity at 25° C. ranging from 1000 to 15 000 mPa·s and an amine number ranging from 2 to 10 mg of KOH per gram of amino silicone; —a surfactant mixture comprising one or more nonionic surfactants, said mixture having an HLB ranging from 10 to 16; and—water; and d) at least 0.01% by weight, relative to the total weight of the composition, of one or more agents chosen from fatty amines, fatty acids, fatty alcohols and mixtures thereof.

The present invention relates to a cosmetic composition comprising at least one anionic surfactant, at least one amphoteric or zwitterionic surfactant, at least one oil-in-water emulsion and at least one agent chosen from a fatty amine, a fatty acid, a fatty alcohol and mixtures thereof, in a particular content.

The invention also relates to the use of said composition for the cosmetic treatment of keratin fibres and to a cosmetic treatment process using this composition.

### TECHNICAL FIELD

In the field of the cosmetic treatment of keratin fibres, notably human keratin fibres such as the hair, and more particularly in the field of hair washing and/or haircare, rinse-out products but also leave-in products are used. These products aim to provide various cosmetic properties, notably smoothness, sheen, softness, suppleness, lightness, a natural feel and good disentangling properties.

Nevertheless, these products lead to compositions with insufficient foam qualities, notably in terms of creaminess and smoothness of the foam. Indeed, the foams are generally less creamy and less dense.

There is thus a need to develop cosmetic compositions that overcome the abovementioned drawbacks without, however, degrading the cosmetic properties.

The Applicant has now discovered that a composition containing at least two different particular surfactants, a particular oil-in-water emulsion and an agent chosen from specific fatty substances in a certain content, makes it possible to obtain products which have excellent foam properties and optimum working qualities (easy application and homogeneous spreading).

In particular, the foam obtained with the compositions according to the invention is very tight (resembling shaving foam) with very fine bubbles of very small size, which leads to the foam qualities mentioned below.

In addition, the compositions according to the invention make it possible to give the hair advantageous cosmetic properties, notably in terms of smoothness, sheen, softness, suppleness, lightness, a natural feel and good disentangling properties.

Thus, the aim of the present invention is to design a cosmetic composition which can afford improved foam qualities, notably in terms of creaminess, smoothness and density, and also having good cosmetic properties, such as smoothness, sheen, softness, suppleness, lightness, a natural feel and good disentangling properties, and also optimum working qualities.

### DISCLOSURE OF THE INVENTION

A subject of the present invention is thus a cosmetic composition comprising:
a) at least one anionic surfactant;
b) at least one amphoteric or zwitterionic surfactant;
c) an oil-in-water emulsion having a particle size D50 of less than 350 nm, the size being expressed on a volume basis, and comprising:
   a silicone mixture comprising (i) one or more polydialkylsiloxanes bearing trialkylsilyl end groups, having a viscosity at 25° C. ranging from 40 000 to 100 000 mPa·s and (ii) one or more amino silicones having a viscosity at 25° C. ranging from 1000 to 15 000 mPa·s and an amine number ranging from 2 to 10 mg of KOH per gram of amino silicone;
   a surfactant mixture comprising one or more nonionic surfactants, said mixture having an HLB ranging from 10 to 16; and
   water; and
d) at least 0.01% by weight, relative to the total weight of the composition, of one or more agents chosen from fatty amines, fatty acids, fatty alcohols and mixtures thereof.

The composition of the invention makes it possible to obtain an abundant foam, which has good creaminess, smoothness and density. This foam can then be easily and uniformly spread on the keratin fibres.

Moreover, the composition of the invention rinses out rapidly without leaving unpleasant residues on the fibres and gives them a natural, clean feel after rinsing. The fibres treated with the composition of the invention also have good cosmetic properties, notably in terms of smoothness, sheen, softness, suppleness, lightness, a natural feel and good disentangling properties.

A subject of the present invention is also a process for the cosmetic treatment, notably the washing, of keratin fibres, in particular human keratin fibres such as the hair, comprising the application to said keratin fibres of the composition according to the invention.

The present invention also relates to the use of a composition according to the invention for washing keratin fibres, in particular human keratin fibres such as the hair.

Other subjects, characteristics, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

### Anionic Surfactants

As indicated previously, the composition according to the invention comprises a) at least one anionic surfactant.

The anionic surfactants a) are different from the fatty acids d).

The term “anionic surfactant” means a surfactant including, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

Preferably, the anionic surfactant(s) are chosen from sulfate-type anionic surfactants.

For the purposes of the present invention, the term “sulfate-type anionic surfactant” means an anionic surfactant including one or more sulfate functions (—OSO₃H or —OSO₃⁻).

Such surfactants may advantageously be chosen from alkyl sulfates, alkyl ether sulfates, alkylamido sulfates, alkylamido ether sulfates, alkylaryl polyether sulfates, monoglyceride sulfates; and also salts thereof and mixtures thereof; the alkyl groups of these compounds notably including from 8 to 30 carbon atoms, preferably from 8 to 26, and more preferentially from 10 to 22 carbon atoms; the aryl group preferably denoting a phenyl or benzyl group; these compounds possibly being polyoxyalkylenated, notably polyoxyethylenated, and preferably including from 1 to 50 ethylene oxide units, and more preferentially from 2 to 10 ethylene oxide units.

Preferably, the anionic surfactant(s) of sulfate type are chosen from:
alkyl sulfates, notably C₈ to C₂₆ and preferably C₁₀ to C₂₂ alkyl sulfates;
alkyl ether sulfates, notably C₈ to C₂₆ and preferably C₁₀ to C₂₂ alkyl ether sulfates, preferably comprising from 2 to 10 ethylene oxide units;
in particular in the form of alkali metal, alkaline-earth metal, ammonium or amino alcohol salts, and mixtures thereof.

When the anionic surfactant(s) of sulfate type are in salt form, said salt may be chosen from alkali metal salts, such as the sodium or potassium salt, ammonium salts, amine salts and in particular amino alcohol salts, and alkaline-earth metal salts, such as the magnesium salt, and mixtures thereof.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferably used.

Preferably, the anionic surfactant(s) of sulfate type are chosen from sodium, triethanolamine, magnesium or ammonium (C₁₀-C₂₂)alkyl sulfates, sodium, triethanolamine, ammonium or magnesium (C₁₀-C₂₂)alkyl ether sulfates, which are oxyethylenated, for example with 1 or 2.2 mol of ethylene oxide, and mixtures thereof.

Better still, the anionic surfactant(s) of sulfate type are chosen from sodium, triethanolamine, ammonium or magnesium (C₁₀-C₂₂)alkyl sulfates or sodium, triethanolamine, ammonium or magnesium (C₁₀-C₂₂)alkyl ether sulfates, such as the compounds sold under the name Texapon Z95P by the company BASF, having the INCI name Sodium lauryl sulfate, or Texapon N701 by the company BASF, having the INCI name Sodium laureth sulfate.

Even more preferentially, the anionic surfactant(s) of sulfate type are chosen from (C₁₀-C₂₂)alkyl ether sulfates and salts thereof, in particular sodium, triethanolamine, ammonium or magnesium (C₁₀-C₂₂)alkyl ether sulfates.

Advantageously, the anionic surfactant(s) of sulfate type are chosen from sodium lauryl sulfate, ammonium lauryl sulfate, sodium lauryl ether sulfate, ammonium lauryl ether sulfate and mixtures thereof, more preferentially chosen from sodium lauryl ether sulfate.

Advantageously, the anionic surfactant(s) may be chosen from sulfonate-type anionic surfactants, carboxylic-type anionic surfactants and mixtures thereof.

For the purposes of the present invention, the term “anionic surfactant of sulfonate type” means an anionic surfactant including one or more sulfonic or sulfonate functions (—SO₃H or —SO₃⁻), which may optionally include one or more carboxylic or carboxylate functions (—COOH or —COO⁻) and which do not include any sulfate functions.

For the purposes of the present invention, the term “anionic surfactant of carboxylate type” means an anionic surfactant including one or more carboxylic or carboxylate functions (—COOH or —COO⁻), and not including any sulfonic or sulfonate functions (—SO₃H or —SO₃⁻) and not including any sulfate functions.

Such anionic surfactants may advantageously be chosen from alkyl sulfonates, alkylamide sulfonates, alkylaryl sulfonates, α-olefin sulfonates, paraffin sulfonates, alkylsulfosuccinates, alkyl ether sulfosuccinates, alkylamide sulfosuccinates, alkyl sulfoacetates, sulfolaurates, N-acyltaurates, acylisethionates, acyllactates, N-acylglycinates, N-acylsarcosinates, N-acylglutamates, alkyl ether carboxylates, alkyl glucose carboxylates, alkyl glucoside tartrates, alkyl glucoside citrates, in salified or non-salified form, and mixtures thereof; the alkyl groups of these compounds notably including from 8 to 30 carbon atoms, preferably from 8 to 26 and more preferentially from 10 to 22 carbon atoms; the aryl group preferably denoting a phenyl or benzyl group; these compounds possibly being polyoxyalkylenated, notably polyoxyethylenated, and then preferably including from 1 to 50 ethylene oxide units, and more preferentially from 2 to 10 ethylene oxide units.

The following are most particularly preferred:
anionic surfactants of the carboxylate type, preferably from among the N—(C₈-C₃₀)acylglutamates, and in particular stearoylglutamates, lauroylglutamates and cocoylglutamates, the N—(C₈-C₃₀)acylsarcosinates, and in particular palmitoylsarcosinates, stearoylsarcosinates, lauroylsarcosinates, cocoylsarcosinates; and mixtures thereof; in particular in the form of alkali metal or alkaline-earth metal, ammonium, amine or amino alcohol salts;
anionic surfactants of sulfonate type, preferably chosen from acylisethionates, and more preferentially from (C₈-C₃₀)acylisethionates used in the form of salts, and better still in the form of alkali metal salts or alkaline-earth metal salts, and in particular sodium or magnesium salts; and
mixtures thereof.

Preferably, the anionic surfactant(s) are chosen from:
alkyl sulfates, notably C₈ to C₂₆ and preferably C₁₀ to C₂₂ alkyl sulfates;
alkyl ether sulfates, notably C₈ to C₂₆ and preferably C₁₀ to C₂₂ alkyl ether sulfates, preferably comprising from 2 to 10 ethylene oxide units;
in particular in the form of alkali metal, alkaline-earth metal, ammonium or amino alcohol salts; and
mixtures thereof.

Preferably, the anionic surfactant(s) are chosen from (C₁₀-C₂₂)alkyl sulfates, notably sodium, triethanolamine, magnesium or ammonium sulfates, (C₁₀-C₂₂)alkyl ether sulfates, notably sodium, triethanolamine, ammonium or magnesium ether sulfates, which are oxyethylenated, for example with 1 or 2.2 mol of ethylene oxide, and mixtures thereof.

Preferentially, the anionic surfactant(s) are chosen from (C₁₀-C₂₂)alkyl ether sulfates and salts thereof, in particular sodium, triethanolamine, ammonium or magnesium (C₁₀-C₂₂)alkyl ether sulfates, and mixtures thereof.

Most particularly, the anionic surfactant(s) are chosen from sodium lauryl sulfate, ammonium lauryl sulfate, sodium lauryl ether sulfate, ammonium lauryl ether sulfate and mixtures thereof, more preferentially sodium lauryl ether sulfate.

The total content of the anionic surfactant(s) present in the composition according to the invention, preferably ranges from 1% to 50% by weight, preferably from 5% to 40% by weight, preferentially from 10% to 30% by weight relative to the total weight of the composition.

Preferably, the composition according to the invention comprises one or more anionic surfactants of the sulfate type. The total content of the anionic surfactant(s) of sulfate type present in the composition according to the invention preferably ranges from 1% to 50% by weight, preferably from 5% to 40% by weight, more preferentially from 10% to 30% by weight relative to the total weight of the composition.

### Amphoteric or Zwitterionic Surfactants

The composition according to the invention also comprises b) at least one amphoteric or zwitterionic surfactant.

In particular, the amphoteric or zwitterionic surfactant(s), which are preferably non-silicone, used in the composition according to the present invention may notably be derivatives of aliphatic secondary or tertiary amines, which are optionally quaternized, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines, and mixtures thereof.

Among the derivatives of secondary or tertiary aliphatic amines, which are optionally quaternized, that can be used, as defined above, mention may also be made of the compounds having the respective structures (I) and (II) below:

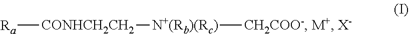

in which formula (I):
   Rₐ represents a C₁₀ to C₃₀ alkyl or alkenyl group derived from an acid RₐCOOH preferably present in hydrolysed coconut kernel oil; preferably, Rₐ represents a heptyl, nonyl or undecyl group;
   R_{b} represents a β-hydroxyethyl group;
   R_{c} represents a carboxymethyl group;
   M⁺ represents a cationic counterion derived from an alkali metal or alkaline-earth metal, such as sodium, an ammonium ion or an ion derived from an organic amine; and
   X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylaryl-sulfonates, in particular methyl sulfate and ethyl sulfate; or alternatively M⁺ and X⁻ are absent;

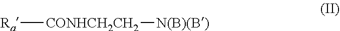

in which formula (II):
   B represents the group —CH₂CH₂OX′;
   B′ represents the group —(CH₂)_{z}Y′, with z=1 or 2;
   X′ represents the group —CH₂COOH, —CH₂—COOZ′, —CH₂CH₂COOH or CH₂CH₂—COOZ′, or a hydrogen atom;
   Y′ represents the group —COOH, —COOZ′ or —CH₂CH(OH)SO₃H or the group CH₂CH(OH)SO₃—Z′;
   Z′ represents a cationic counterion derived from an alkali metal or alkaline-earth metal, such as sodium, an ammonium ion or an ion derived from an organic amine;
   R_{a′} represents a C₁₀ to C₃₀ alkyl or alkenyl group of an acid R_{a′}—COOH which is preferably present in coconut kernel oil or in hydrolysed linseed oil, preferably R_{a′} an alkyl group, notably a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (III):

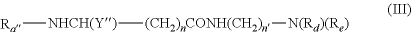

in which formula (III):
   Y″ represents the group —COOH, —COOZ″ or —CH₂—CH(OH)SO₃H or the group CH₂CH(OH)SO₃—Z″;
   R_{d} and Rₑ, independently of each other, represent a C₁ to C₄ alkyl or hydroxyalkyl radical;
   Z″ represents a cationic counterion derived from an alkali metal or alkaline-earth metal, such as sodium, an ammonium ion or an ion derived from an organic amine;
   R_{a″} represents a C₁₀ to C₃₀ alkyl or alkenyl group of an acid R_{a″}—COOH which is preferably present in coconut kernel oil or in hydrolysed linseed oil; and
   n and n′ denote, independently of each other, an integer ranging from 1 to 3.

Among the compounds of formula (I), mention may be made of the compound classified in the CTFA dictionary under the name sodium diethylaminopropyl cocoaspartamide and sold by the company Chimex under the name Chimexane HB.

These compounds may be used alone or as a mixture.

Among the amphoteric or zwitterionic surfactants mentioned above, use is advantageously made of (C₈-C₂₀)alkylbetaines, such as cocoyl betaine (C₈-C₂₀)alkylamido(C₃-C₈)alkylbetaines, such as cocamidopropylbetaine, (C₈-C₂₀)alkylamphoacetates, (C₈-C₂₀)alkylamphodiacetates and mixtures thereof; and preferably (C₈-C₂₀)alkylbetaines, (C₈-C₂₀)alkylamido(C₃-C₈)alkylbetaines and mixtures thereof.

Preferentially, the amphoteric or zwitterionic surfactant(s) are chosen from (C₈-C₂₀)alkylbetaines, (C₈-C₂₀)alkylamido(C₃-C₈)alkylbetaines and mixtures thereof, better still from (C₈-C₂₀)alkylamido(C₃-C₈)alkylbetaines and mixtures thereof.

The total content of the amphoteric or zwitterionic surfactant(s) present in the composition according to the invention preferably ranges from 0.1% to 30% by weight, more preferentially from 0.5% to 20% by weight, better still from 1% to 15% by weight, better still from 1% to 10% by weight, even better still from 1% to 6% by weight relative to the total weight of the composition.

### Oil-In-Water Emulsion

The composition according to the invention also comprises c) at least one oil-in-water emulsion having a particle size D50 of less than 350 nm, the size being expressed on a volume basis, and comprising:
a silicone mixture comprising (i) one or more polydialkylsiloxanes bearing trialkylsilyl end groups, having a viscosity at 25° C. ranging from 40 000 to 100 000 mPa·s and (ii) one or more amino silicones having a viscosity at 25° C. ranging from 1000 to 15 000 mPa·s and an amine number ranging from 2 to 10 mg of KOH per gram of amino silicone;
a surfactant mixture comprising one or more nonionic surfactants, said mixture having an HLB ranging from 10 to 16; and
   water.

In the oil-in-water, or silicone-in-water, emulsion according to the invention, a liquid phase (the dispersed phase) is advantageously dispersed in another liquid phase (the continuous phase); in the present invention, the mixture of silicones (or silicone mixture), or silicone phase, is dispersed in the aqueous continuous phase.

The silicone mixture comprises one or more polydialkylsiloxanes bearing trialkylsilyl end groups, preferably of formula (I): R′3SiO(R′2SiO)pSiR′3 (I), in which:
R′, which may be identical or different, is a monovalent hydrocarbon-based radical containing from 1 to 18 carbon atoms, preferably from 1 to 6 carbon atoms, better still from 1 to 3 carbon atoms, even better still a methyl radical, and
p is an integer ranging from 500 to 2000, better still from 1000 to 2000.

The polydialkylsiloxanes bearing trialkylsilyl end groups according to the invention have a viscosity ranging from 40 000 to 100 000 mPa·s, preferably 100 000 being excluded at 25° C. preferably ranging from 40 000 to 70 000 mPa·s at 25° C., better still from 51 000 to 70 000 mPa·s at 25° C.

The polydialkylsiloxanes bearing trialkylsilyl end groups according to the invention are preferably linear, but they may comprise, in addition to the R′₂SiO_{2/2} units (D-units), additional RSiO_{3/2} units (T-units) and/or SiO_{4/2} units (Q-units), in which R′, which may be identical or different, is a C₁-C₁₈ monovalent hydrocarbon-based radical.

Preferably, in formula (I), R′, which may be identical or different, is:
an alkyl, preferably C₁-C₂₈ alkyl, radical, such as the radicals: methyl, ethyl, n-propyl, isopropyl, 1-n-butyl, 2-n-butyl, isobutyl, tert-butyl, n-pentyl, isopentyl, neopentyl, tert-pentyl, hexyl and notably n-hexyl, heptyl and notably n-heptyl, octyl and notably n-octyl, isooctyl, 2,2,4-trimethylpentyl; nonyl and notably n-nonyl; decyl and notably n-decyl; dodecyl and notably n-dodecyl; octadecyl and notably n-octadecyl;
an alkenyl radical such as vinyl and allyl;
   a cycloalkyl radical such as cyclopentyl, cyclohexyl, cycloheptyl and methylcyclohexyl;
an aryl radical such as phenyl, naphthyl, anthryl and phenanthryl;
an alkaryl radical such as o-, m- and p-tolyl; xylyl and ethylphenyl radicals;
an aralkyl radical such as benzyl and phenylethyl.

Preferentially, R′ is a methyl radical.

Preferably, the polydialkylsiloxanes bearing trialkylsilyl end groups are polydimethylsiloxanes (PDMSs) bearing trialkylsilyl end groups.

The silicone mixture also comprises one or more amino silicones, preferably of formula (II):

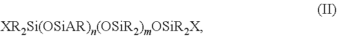

in which:
   R, which may be identical or different, is a monovalent hydrocarbon-based radical containing from 1 to 18 carbon atoms, preferably from 1 to 6 carbon atoms, better still from 1 to 3 carbon atoms, even better still a methyl radical;
   X, which may be identical or different, represents R or a hydroxyl (OH) or a C₁-C₆ alkoxy group; preferably X is R, i.e. a monovalent hydrocarbon-based radical containing from 1 to 18 carbon atoms, preferably from 1 to 6 carbon atoms, better still from 1 to 3 carbon atoms, even better still a methyl radical;
   A is an amino radical of formula —R′—[NR²—R³—]_{X}NR²2, or the protonated form of this amino radical, with
   R¹ representing a C₁-C₆ alkylene radical, preferably a —CH₂CH₂CH₂— or —CH₂CH(CH₃)CH₂— radical;
   R², which may be identical or different, being a hydrogen atom or a C₁-C₄ alkyl radical, preferably a hydrogen atom;
   R³ being a C₁-C₆ alkylene radical, preferably a —CH₂CH₂— radical;
   x is 0 or 1;
   m and n are integers such that m+n ranges from 50 to 1000, better still from 50 to 600.

Preferably, A is an amino radical of formula —R′—[NR²—R³—]ₓNR²₂, or the protonated form of this amino radical, with R¹ being —CH₂CH₂CH₂— or —CH₂CH(CH₃)CH₂—, R² being hydrogen atoms, R³ being —CH₂CH₂— and x being equal to 1.

Preferably, R, which may be identical or different, is:
an alkyl, preferably C₁-C₂₈ alkyl, radical, such as the radicals: methyl, ethyl, n-propyl, isopropyl, 1-n-butyl, 2-n-butyl, isobutyl, tert-butyl, n-pentyl, isopentyl, neopentyl, tert-pentyl, hexyl and notably n-hexyl, heptyl and notably n-heptyl, octyl and notably n-octyl, isooctyl, 2,2,4-trimethylpentyl; nonyl and notably n-nonyl; decyl and notably n-decyl; dodecyl and notably n-dodecyl; octadecyl and notably n-octadecyl;
an alkenyl radical such as vinyl and allyl;
   a cycloalkyl radical such as cyclopentyl, cyclohexyl, cycloheptyl and methylcyclohexyl;
an aryl radical such as phenyl, naphthyl, anthryl and phenanthryl;
an alkaryl radical such as the radicals o-, m- and p-tolyl; xylyl, ethylphenyl;
an aralkyl radical such as benzyl and phenylethyl.

Preferentially, R is a methyl radical.

The amino silicones according to the invention have a viscosity at 25° C. ranging from 1000 to 15 000 mPa·s, preferably from 1500 to 15 000 mPa·s.

The amino silicones according to the invention have an amine number ranging from 2 to 10 mg of KOH per gram of amino silicone, preferably from 3.5 to 8 mg. The molar percentage of amine function is preferably between 0.3 mol % and 8 mol %.

As examples of amino silicones, mention may be made of amino silicones bearing trialkylsilyl end groups; preferably aminoethylaminopropylmethylsiloxanes bearing trialkylsilyl end groups, better still copolymers of aminoethylaminopropylmethylsiloxane bearing trialkylsilyl end groups/dimethylsiloxane.

The amino radical A may be partially or totally protonated, for example by addition of acids to the amino silicone, so as to obtain the salified form of said amino radical.

As acids that may be used, mention may be made of linear or branched carboxylic acids containing from 3 to 18 carbon atoms, such as formic acid, acetic acid, propionic acid, butyric acid, pivalic acid, sorbic acid, benzoic acid or salicylic acid. Preferably, the acids may be used in a proportion of from 0.1 to 2.0 mol per mole of amino radical A in the amino silicone of formula (II).

The silicone mixture preferably comprises (i) one or more polydialkylsiloxanes bearing trialkylsilyl end groups, having a viscosity at 25° C. ranging from 40 000 to 100 000 mPa·s, preferably 100 000 mPa·s being excluded, in an amount of from 70% to 90% by weight, preferably from 75% to 85% by weight, relative to the total weight of the silicone mixture, and (ii) one or more amino silicones having a viscosity at 25° C. ranging from 1000 to 15 000 mPa·s and an amine number ranging from 2 to 10 mg of KOH per gram of amino silicone, in an amount of from 10% to 30% by weight, notably from 15% to 25% by weight, relative to the total weight of the silicone mixture.

The oil-in-water emulsion also comprises a surfactant mixture which comprises one or more nonionic surfactants; said surfactant mixture may optionally comprise one or more cationic surfactants.

Said surfactant mixture has an HLB ranging from 10 to 16.

The nonionic surfactants that may be used may be chosen from alcohols, α-diols and (C₁-20)alkylphenols, these compounds being polyethoxylated and/or polypropoxylated and/or polyglycerolated, the number of ethylene oxide and/or propylene oxide groups possibly ranging from 1 to 100, and the number of glycerol groups possibly ranging from 2 to 30; or else these compounds comprising at least one fatty chain including from 8 to 30 carbon atoms and notably from 16 to 30 carbon atoms.

Mention may also be made of condensates of ethylene oxide and of propylene oxide with fatty alcohols; polyethoxylated fatty amides preferably containing from 2 to 30 ethylene oxide units, polyglycerolated fatty amides including on average from 1 to 5, and in particular from 1.5 to 4, glycerol groups; ethoxylated fatty acid esters of sorbitan preferably containing from 2 to 40 ethylene oxide units, fatty acid esters of sucrose, polyoxyalkylenated and preferably polyoxyethylenated fatty acid esters containing from 2 to 150 mol of ethylene oxide, including oxyethylenated plant oils, N—(C₆-C₂₄ alkyl)glucamine derivatives, amine oxides such as (C₁₀-C₁₄ alkyl)amine oxides or N—(C₁₀-C₁₄ acyl)aminopropylmorpholine oxides.

Mention may also be made of nonionic surfactants of alkyl(poly)glycoside type, notably represented by the following general formula: R₁O—(R₂O)ₜ-(G)v in which:
R₁ represents a linear or branched alkyl or alkenyl radical including 6 to 24 carbon atoms and notably 8 to 18 carbon atoms, or an alkylphenyl radical of which the linear or branched alkyl radical includes 6 to 24 carbon atoms and notably 8 to 18 carbon atoms;
R₂ represents an alkylene radical including 2 to 4 carbon atoms,
G represents a sugar unit including 5 to 6 carbon atoms,
t denotes a value ranging from 0 to 10 and preferably from 0 to 4,
v denotes a value ranging from 1 to 15 and preferably from 1 to 4.
   Preferably, the alkyl(poly)glycoside surfactants are compounds of the formula described above in which:
R₁ denotes a linear or branched, saturated or unsaturated alkyl radical including from 8 to 18 carbon atoms,
R₂ represents an alkylene radical including 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C₈/C₁₆ alkyl (poly)glucosides of 1-4 type, and notably decyl glucosides and caprylyl/capryl glucosides, are most particularly preferred.

Among the commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or the products sold by the company Chem Y under the name AG10 LK.

Preferably, use is made of C₈/C₁₆ alkyl (poly)glycosides of 1-4 type, notably as an aqueous 53% solution, such as those sold by Cognis under the reference Plantacare® 818 UP.

The mono- or polyglycerolated surfactants preferably comprise an average number of glycerol groups ranging from 1 to 30, notably from 1 to 10, better still from 1.5 to 5. They preferably correspond to one of the following formulae:

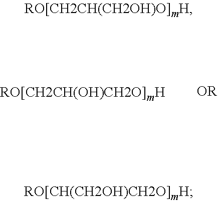

in which:
   R represents a saturated or unsaturated, linear or branched hydrocarbon-based (notably alkyl or alkenyl) radical including 8 to 40 carbon atoms, notably 10 to 30 carbon atoms, optionally comprising one or more heteroatoms such as O and N; and
   m is an integer ranging from 1 to 30, preferably from 1 to 10, better still from 1.5 to 6.

In particular, R may comprise one or more hydroxyl and/or ether and/or amide groups. Preferably, R is a mono- or polyhydroxylated C₁₀-C₂₀ alkyl or alkenyl radical.

Mention may be made of polyglycerolated (3.5 mol) hydroxylauryl ether, such as the product Chimexane® NF from Chimex.

Mention may also be made of (poly)ethoxylated fatty alcohols preferably comprising one or more saturated or unsaturated, linear or branched hydrocarbon-based chains comprising 8 to 30 carbon atoms, preferably from 12 to 22 carbon atoms, optionally substituted with one or more hydroxyl (OH) groups, notably 1 to 4 hydroxyl groups.

When the chain is unsaturated, it may comprise one to three conjugated or non-conjugated carbon-carbon double bonds.
The (poly)ethoxylated fatty alcohols preferably correspond to formula (II):

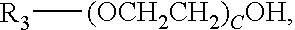

in which:
R3 represents a linear or branched alkyl or alkenyl radical including from 8 to 40 carbon atoms and notably 8 to 30 carbon atoms, optionally substituted with one or more, notably 1 to 4, hydroxyl groups; and
c is an integer ranging from 1 to 200, notably from 2 to 150, or even from 4 to 50 and even better still from 8 to 30.
   The (poly)ethoxylated fatty alcohols are more particularly fatty alcohols comprising from 8 to 22 carbon atoms, oxyethylenated with 1 to 30 mol of ethylene oxide (1 to 30 EO); mention may in particular be made of lauryl alcohol 2 EO; lauryl alcohol 3 EO; decyl alcohol 3 EO; decyl alcohol 5 EO and oleyl alcohol 20 EO.

The nonionic surfactants may advantageously be chosen from:
(i) (poly)oxyalkylenated, notably (poly)ethoxylated, fatty alcohols, and in particular those of formula: R₃—(OCH₂CH₂)_{c}OH in which:
   R3 represents a linear or branched alkyl or alkenyl radical including from 8 to 40 carbon atoms and notably 8 to 30 carbon atoms, optionally substituted with one or more, notably 1 to 4, hydroxyl groups; and
   c is an integer ranging from 1 to 200, notably from 2 to 150, or even from 4 to 50 and even better still from 8 to 20.
(ii) (poly)oxyalkylenated (C₈-C₃₂)alkyl phenyl ethers, notably comprising from 1 to 200, better still from 1 to 30 mol of ethylene oxide;
(iii) polyoxyalkylenated esters of C₈-C₃₂ fatty acids and of sorbitan, notably polyoxyethylenated esters of C₈-C₃₂ fatty acids and of sorbitan, preferably containing from 2 to 40 ethylene oxide units, better still from 2 to 20 ethylene oxide (EO) units; in particular polyoxyethylenated esters of C₁₀-C₂₄ fatty acids and of sorbitan, preferably containing from 2 to 40 ethylene oxide units, better still from 2 to 20 ethylene oxide (EO) units; and
(iv) polyoxyethylenated esters of C₈-C₃₂ fatty acids, preferably containing from 2 to 150 ethylene oxide units; notably polyoxyethylenated esters of C₁₀-C₂₄ fatty acids, notably comprising 2 to 150 ethylene oxide (EO) units.

The nonionic surfactants may advantageously be chosen from alkyl ethers and alkyl esters of polyalkylene glycol, notably of polyethylene glycol.

Mention may in particular be made of:
polyethylene glycol octyl ether; polyethylene glycol lauryl ether; polyethylene glycol tridecyl ether; polyethylene glycol cetyl ether; polyethylene glycol stearyl ether; and
most particularly trideceth-3, trideceth-10 and steareth-6;
polyethylene glycol nonylphenyl ether; polyethylene glycol dodecylphenyl ether;
polyethylene glycol cetylphenyl ether; polyethylene glycol stearylphenyl ether;
polyethylene glycol sorbitan monostearate, polyethylene glycol sorbitan monooleate;
polyethylene glycol stearate, and notably PEG100 stearate.

Better still, the nonionic surfactants may be chosen from Steareth-6, PEG100 stearate, trideceth-3 and trideceth-10, and mixtures thereof; most particularly, a mixture comprising these four nonionic surfactants.

The surfactant mixture may optionally comprise one or more cationic surfactants, which may be chosen from tetraalkylammonium, tetraarylammonium and tetraalkylarylammonium salts, notably halides, and most particularly from cetrimonium or behentrimonium salts, notably halides, better still chlorides.

The oil-in-water emulsion preferably comprises the surfactant mixture in a total amount ranging from 5% to 15% by weight, notably from 8% to 15% by weight, even better still from 10% to 12% by weight, relative to the total weight of the emulsion.

The oil-in-water emulsion preferably comprises the nonionic surfactant(s) in a total amount ranging from 5% to 15% by weight, notably from 8% to 15% by weight, even better still from 10% to 12% by weight, relative to the total weight of the emulsion.

The oil-in-water emulsion preferably comprises the cationic surfactant(s), when they are present, in a total amount ranging from 0.5% to 1.5% by weight relative to the total weight of the emulsion.

The oil-in-water emulsion preferably comprises the silicone mixture in a total amount ranging from 40% to 60% by weight, notably from 45% to 55% by weight, relative to the total weight of the emulsion.

The oil-in-water emulsion preferably comprises the polydialkylsiloxane(s) bearing trialkylsilyl end groups in a total amount ranging from 35% to 45% by weight, notably from 38% to 42% by weight, relative to the total weight of the emulsion.

The oil-in-water emulsion preferably comprises the amino silicone(s) in a total amount ranging from 5% to 15% by weight, notably from 8% to 12% by weight, relative to the total weight of the emulsion.

The oil-in-water emulsion preferably comprises water in a total amount ranging from 25% to 50% by weight, notably from 30% to 45% by weight, even better still from 35% to 42% by weight, relative to the total weight of the emulsion.

The oil-in-water emulsion may also comprise a preserving agent, such as phenoxyethanol, in an amount ranging from 0.5% to 1% by weight relative to the total weight of the emulsion.

A process for preparing the oil-in-water emulsion preferably comprises:
a step of mixing one or more polydialkylsiloxanes bearing trialkylsilyl end groups, having a viscosity at 25° C. ranging from 40 000 to 100 000 mPa·s, preferably 100 000 mPa·s being excluded, and one or more amino silicones having a viscosity at 25° C. ranging from 1000 to 15 000 mPa·s and an amine number ranging from 2 to 10 mg of KOH per gram of amino silicone; at a temperature of from 15° C. to 40° C., notably at 25° C., so as to obtain a fluid silicone mixture; and then
a step of adding a surfactant mixture comprising one or more nonionic surfactants, said mixture having an HLB ranging from 10 to 16, to said fluid silicone mixture, so as to obtain an emulsified silicone mixture; and then
a step of homogenizing said emulsified silicone mixture, followed by
a step of adding water, notably demineralized water, preferentially in steps, so as to obtain an oil-in-water emulsion having a particle size D50 of less than 350 nm.
   The preparation process may also comprise an additional step of adding one or more preserving agents.

The pH of the oil-in-water emulsion is generally between 4 and 6.

The oil-in-water emulsion has a particle size D50 of less than 350 nm, notably between 100 and 300 nm, better still between 150 and 250 nm, even better still between 160 and 200 nm, the size being expressed on a volume basis.

This size corresponds to the average hydrodynamic particle diameter. The particle size D50 is expressed on a volume basis. It may be measured using a ZetaSizer device from Malvern, UK, model Nano-ZS, based on the “Photon Correlation Spectroscopy (PCS)” method.

### Method for Measuring the Particle Size

The particle size of the emulsion is measured using a ZetaSizer device from Malvern, UK, model Nano-ZS, based on the “Photon Correlation Spectroscopy (PCS)” method.

The particle size D50 is measured when the evaluation algorithm is “cumulant analysis”.

0.5 g of the emulsion is placed in a 250 ml beaker, 100 ml of demineralized water are added and mixing is performed so as to obtain the solution to be tested. The solution to be tested is placed in the measuring cuvette (or cell) and introduced into the measuring device.

The size D₅₀ corresponds to the particle diameter value at 50% in cumulative distribution.

For example, if D₅₀=170 nm, this means that 50% of the particles have a size of greater than 170 nm, and that 50% of the particles have a size of less than 170 nm.

It should be recalled that this distribution is on a volume basis.

### Method for Measuring the Viscosity

The viscosities, notably of the silicone compounds, are measured at 25° C., 1 atm.

To measure viscosities of between 1000 and 40 000 mPa·s at 25° C., use may be made of an Anton Paar rheometer, model MCR101, cylinder geometry, single gap: CC27 spindle, shear rate 1 s⁻¹ for 2 minutes, at 25° C.

To measure viscosities of between 40 000 and 100 000 mPa·s at 25° C., use may be made of an Anton Paar rheometer, model MCR101, 25-6 cone (cone-plate geometry, 25 mm in diameter/6° cone); Zero gap, shear rate 1 s⁻¹ for 2 minutes, at 25° C.

Three measurements are performed for each sample, and the viscosity value is taken at 60 seconds. The MCR Rheometer Series products operate according to the USP convention (US Pharmacopeia Convention, 912—Rotational Rheometer methods).

### Method for Measuring the Amine Number

The amine number may be measured by acid-base titration, using a potentiometer [Make: Veego; model VPT-MG].

0.6 g of the sample is placed in a 500 ml beaker and a 1:1 toluene-butanol mixture is added, and the whole is then mixed.

The solution is titrated with 0.1 N HCl solution. Determination of the zero value (V_{blank}) is also performed with the 1:1 toluene-butanol mixture alone.

The amine number is calculated by means of the formula:

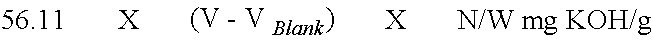

of sample
with V=volume of HCl required (in ml), V_{Blank}=volume of HCl required for the zero value (in ml); N=normality of HCl, i.e. 0.1, and W=mass of the sample (in g).

### HLB Values

The term “HLB” relates to the hydrophilic-lipophilic balance of a surfactant. It may be measured experimentally or calculated.

In the present patent application, the HLB values are the values at 25° C.

The HLB values may be calculated by means of the following equation: HLB=(E+P)/5, in which E is the weight percentage of oxyethylene and P is the weight percentage of polyol, as described in the Griffin publication, J. Soc. Cosm. Chem. 1954 (volume 5, No. 4), pages 249-256.

The HLB values may also be determined experimentally according to the book by Puisieux and Seiller, entitled “Galenica 5: Les systèmes disperses [Galenics 5: Dispersed systems]—Volume I—Agents de surface et émulsions [Surface agents and emulsions]—Chapter IV—Notions de HLB et de HLB critique [Notions of HLB and of critical HLB], pages 153-194—paragraph 1.1.2. Détermination de HLB par voie expérimentale [Experimental determination of HLB], pages 164-180”.

Preferably, the HLB values that will be taken into account are those obtained by calculation, notably in the following manner: “calculated HLB”=20×(molar mass of the hydrophilic part/total molar mass).

Thus, for an oxyethylenated fatty alcohol, the hydrophilic part corresponds to the oxyethylene units fused to the fatty alcohol and the “calculated HLB” then corresponds to the “HLB according to Griffin”.

For an ester or an amide, the hydrophilic part is generally defined as being beyond the carbonyl group, starting from the fatty chain(s).

The HLB values of nonionic surfactants may also be calculated by means of the Davies formula, as described in Davies JT (1957), “A quantitative kinetic theory of emulsion type, I. Physical chemistry of the emulsifying agent”, Gas/Liquid and Liquid/Liquid Interface (Proceedings of the International Congress of Surface Activity): 426-438.

According to this formula, the HLB value is obtained by adding the hydrophilic/hydrophobic contribution linked to the constituent groups of the surfactant:

$$H L B = ( number of hydrophilic groups ) - n ( number of groups per {CH}_{2} group ) + 7.$$

The HLB values of some cationic surfactants are given in Table IV, in “Cationic emulsifiers in cosmetics”, Godfrey, J. Soc. Cosmetic Chemists (1966) 17, pages 17-27.

When two surfactants A and B, of known HLB values, are mixed, the HLB_{Mix} corresponds to the HLB of the mixture and can be expressed by the following equation:

HLB_{Mix}=(W_{A}HLB_{A}+W_{B}HLB_{B})/(W_{A}+W_{B}),
in which W_{A} is the amount (weight) of the first surfactant A and W_{B} the amount of the second surfactant B, and HLB_{A} and HLB_{B} are the HLB values of the surfactant A and of the surfactant B.

Preferably, the composition according to the invention comprises the oil-in-water emulsion c) in a total amount ranging from 0.1% to 10% by weight, better still from 0.2% to 8% by weight, preferentially from 0.5% to 6% by weight, relative to the total weight of the composition.

Preferably, the composition according to the invention comprises the oil-in-water emulsion c) in a total amount ranging from 0.1% to 10% by weight, better still from 0.2% to 8% by weight, preferentially from 0.5% to 6% by weight, relative to the total weight of the composition, and the emulsion has a solids (or active material) content of between 40% and 60% by weight, notably 45% to 55% by weight, relative to the total weight of the emulsion.

Preferably, the composition according to the invention comprises the polydialkylsiloxane(s) bearing trialkylsilyl end groups, having a viscosity at 25° C. ranging from 40 000 to 100 000 mPa·s, in a total amount ranging from 0.04% to 4% by weight, better still from 0.08% to 3.2% by weight, preferentially from 0.2% to 2.4% by weight, relative to the total weight of the composition.

Preferably, the composition according to the invention comprises the amino silicone(s) having a viscosity at 25° C. ranging from 1000 to 15 000 mPa·s and an amine number of from 2 to 10 mg KOH per gram of amino silicone in a total amount of from 0.02% to 2% by weight, better still from 0.04% to 1.6% by weight, preferentially from 0.1% to 1.2% by weight, relative to the total weight of the composition.

### Particular Agent

As previously indicated, the composition according to the invention also comprises at least 0.01% by weight, relative to the total weight of the composition, of one or more agents chosen from fatty amines, fatty acids, fatty alcohols and mixtures thereof.

Thus, said agent is a particular fatty substance chosen from a fatty amine, a fatty acid, a fatty alcohol and mixtures thereof.

The term “fatty substance” means an organic compound that is insoluble in water at standard temperature (25° C.) and at atmospheric pressure (760 mmHg, i.e. 1.013×105 Pa), i.e. with a solubility of less than 5%, preferably of less than 1% and even more preferentially of less than 0.1%.

Fatty substances are generally soluble in organic solvents under the same temperature and pressure conditions, for instance chloroform, ethanol, benzene, liquid petroleum jelly or decamethylcyclopentasiloxane.

The term “fatty amine” means a compound comprising at least one optionally (poly)oxyalkylenated primary, secondary or tertiary amine function, or salts thereof and comprising at least one C₆-C₃₀, preferably C₈-C₂₈, hydrocarbon-based chain.

Preferably, the fatty amines that are useful according to the invention are not (poly)oxyalkylenated.

Advantageously, the fatty amines are chosen from the fatty amidoamines. Preferably, the fatty chain of the fatty amidoamines may be borne by the amine group or by the amido group.

The term “amidoamine” means a compound comprising at least one amide function and at least one primary, secondary or tertiary amine function.

The term “fatty amidoamine” means an amidoamine comprising, in general, at least one C₆-C₃₀ and notably C₈-C₂₈ hydrocarbon-based chain. Preferably, the fatty amidoamines that are useful according to the invention are not quaternized.

Preferably, the fatty amidoamines that are useful according to the invention are not (poly)oxyalkylenated.

Among the fatty amidoamines that are useful according to the invention, mention may be made of the amidoamines of formula (B) below:

RCONHR″N(R′)₂, in which: (B)
R represents a substituted or unsubstituted, linear or branched, saturated or unsaturated monovalent hydrocarbon-based radical containing from 5 to 29 carbon atoms, preferably from 7 to 23 carbon atoms, and in particular a linear or branched C₅-C₂₉ and preferably C₇-C₂₃ alkyl radical, or a linear or branched C₅-C₂₉ and preferably C₇-C₂₃ alkenyl radical;
R″ represents a divalent hydrocarbon-based radical containing less than 6 carbon atoms, preferably 2 to 4 carbon atoms and better still 3 carbon atoms; and
R′, which may be identical or different, represent a linear or branched, saturated or unsaturated and substituted or unsubstituted monovalent hydrocarbon-based radical containing less than 6 carbon atoms, preferably from 1 to 4 carbon atoms, preferably a methyl radical.

The fatty amidoamines of formula (B) are chosen, for example, from oleamidopropyl dimethylamine, stearamidopropyl dimethylamine sold by Inolex Chemical Company under the name Lexamine S13, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, avocadoamidopropyl dimethylamine, cocamidopropyl dimethylamine, minkamidopropyl dimethylamine, oatamidopropyl dimethylamine, sesamidopropyl dimethylamine, tallamidopropyl dimethylamine, olivamidopropyl dimethylamine, palmitamidopropyl dimethylamine, stearamidoethyldiethylamine, brassicamidopropyl dimethylamine, and mixtures thereof.

Preferably, the fatty amidoamines are chosen from oleamidopropyl dimethylamine, stearamidopropyl dimethylamine, brassicamidopropyl dimethylamine and mixtures thereof, more preferentially stearamidopropyl dimethylamine.

Preferably, the fatty acids according to the invention are chosen from linear or branched, saturated or unsaturated fatty acids including from 8 to 30 carbon atoms, better still from 12 to 22 carbon atoms.

Preferably, they are chosen from linear saturated solid fatty acids containing from 8 to 30 carbon atoms, better from 12 to 22 carbon atoms, the C₈-30, preferably C₁₂-22, alkyl chain possibly being substituted with at least one hydroxyl group.

Preferably the fatty acids are monoacids, notably of formula R—COOH with R representing a linear or branched, saturated or unsaturated, preferably linear and saturated, C₈-C₃₀, better still C₁₀-C₂₆, even better still C₁₂-C₂₂, alkyl group.

Preferably, the fatty acid(s) are chosen from lauric acid, myristic acid, palmitic acid, stearic acid, 12-hydroxystearic acid, behenic acid and mixtures thereof, better still lauric acid, stearic acid, 12-hydroxystearic acid and behenic acid, even more preferentially lauric acid.

Preferably, the fatty alcohols are chosen from saturated or unsaturated, linear or branched fatty alcohols including from 8 to 30 carbon atoms, better from 12 to 22 carbon atoms, preferably from solid, linear saturated fatty alcohols containing from 8 to 30 carbon atoms, better still from 12 to 22 carbon atoms.

Preferably, the fatty alcohols are monoalcohols, advantageously C₈-C₃₀, better still C₁₀-C₂₆ and even better still C₁₂-C₂₂ monoalcohols.

Preferably, the fatty alcohol(s) are chosen from lauryl alcohol, myristic alcohol, cetearyl alcohol, stearyl alcohol, behenyl alcohol and mixtures thereof, more preferentially from lauryl alcohol and cetearyl alcohol.

Preferably, the composition comprises one or more fatty amines, notably one or more fatty amidoamines, even better still one or more fatty amidoamines of formula (B).

Advantageously, the total content of agent(s) chosen from fatty amines, fatty acids, fatty alcohols and mixtures thereof ranges from 0.01% to 5% by weight, preferably from 0.1% to 3% by weight, more preferentially from 0.3% to 2% by weight, better still from 0.4% to 1% by weight relative to the total weight of the composition.

### Organic Solvents

The composition according to the invention may comprise one or more organic solvents.

Preferably, the organic solvent(s) are chosen from linear or branched monoalcohols containing from 1 to 6 carbon atoms, preferentially from 1 to 4 carbon atoms, polyols and notably those comprising from 2 to 6 carbon atoms and 2 to 4 hydroxyl groups, polyethylene glycols, aromatic alcohols, and mixtures thereof.

As examples of organic solvents that may be used according to the invention, mention may notably be made of ethanol, propanol, butanol, isopropanol, isobutanol, propylene glycol, dipropylene glycol, butylene glycol, glycerol, benzyl alcohol and phenoxyethanol, and mixtures thereof.

The organic solvent(s) that may be used according to the invention may be chosen, alone or as a mixture, from linear or branched monoalcohols containing from 1 to 4 carbon atoms, preferably from ethanol, isopropanol, and mixtures thereof; polyols and more preferentially from glycerol, propylene glycol, and mixtures thereof.

In a preferred embodiment of the invention, the composition comprises one or more organic solvents, preferentially one or more C₂-C₄ polyols and comprising 2 to 4 OH, better still glycerol.

The total content of the organic solvent(s), when they are present in the composition according to the invention, is preferably less than or equal to 20% by weight, more preferentially less than or equal to 15% by weight, and better still ranges from 0.1% to 12% by weight, relative to the total weight of the composition.

Advantageously, the composition may comprise water, notably in an amount of at least 60% by weight, better still of at least 65% by weight, even better still of at least 70% by weight, relative to the total weight of the composition. In particular, it may comprise water in a total amount ranging from 60% to 98% by weight, notably from 65% to 95% by weight, still better from 70% to 90% by weight relative to the total weight of the composition.

### Cationic Polymers

The composition according to the invention may also comprise one or more cationic polymers.

For the purposes of the present invention, the term “cationic polymer” denotes any non-silicone (not comprising any silicon atoms) polymer containing cationic groups and/or groups that can be ionized into cationic groups and not containing any anionic groups and/or groups that can be ionized into anionic groups.

The cationic polymers are not silicone-based (they do not comprise any Si—O units).

The cationic polymers may be associative or non-associative.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:
(1) homopolymers or copolymers derived from acrylic or methacrylic esters or amides and including at least one of the units having the following formulae:

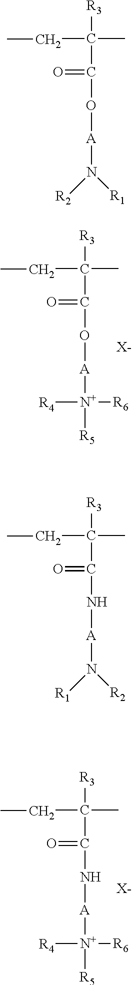

in which formulae:
   R₃, which may be identical or different, denote a hydrogen atom or a CH₃ radical;
   A, which may be identical or different, represent a linear or branched divalent alkyl group of 1 to 6 carbon atoms, preferably 2 or 3 carbon atoms, or a hydroxyalkyl group of 1 to 4 carbon atoms;
   R₄, R₅ and R₆, which may be identical or different, represent an alkyl group containing from 1 to 18 carbon atoms or a benzyl radical, and preferably an alkyl group containing from 1 to 6 carbon atoms;
   R₁ and R₂, which may be identical or different, represent a hydrogen atom or an alkyl group containing from 1 to 6 carbon atoms, preferably methyl or ethyl; and
   X denotes an anion derived from a mineral or organic acid, such as a methosulfate anion or a halide such as chloride or bromide.

The copolymers of the family (1) may also contain one or more units derived from comonomers which may be chosen from the family of acrylamides, methacrylamides, diacetone acrylamides, acrylamides and methacrylamides substituted on the nitrogen with lower alkyls (C₁-C₄), acrylic acids or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, such as the products sold under the name Bina Quat P 100 by the company Ciba Geigy,
the copolymer of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the products sold under the name Styleze CC 10 by ISP;
quaternized vinylpyrrolidone/dimethylaminopropylmethacrylamide copolymers such as the product sold under the name Gafquat HS 100 by the company ISP;
polymers, preferably crosslinked polymers, of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.
(2) cationic polysaccharides, notably cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethylcellulose that have reacted with an epoxide substituted with a trimethylammonium group, for instance Polyquaternium-10.

Cationic cellulose copolymers or cellulose derivatives grafted with a water-soluble quaternary ammonium monomer are notably described in U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt, for instance Polyquaternium-4. The commercial products corresponding to this definition are more particularly the products sold under the names Celquat L 200 and Celquat H 100 by the company National Starch.

Among the cationic cellulose derivatives, use may also be made of cationic associative celluloses, which may be chosen from quaternized cellulose derivatives, and in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (Ib):

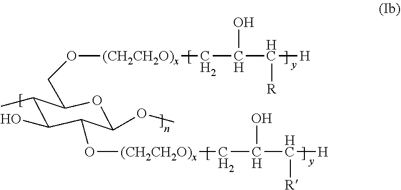

in which:
   R represents an ammonium group RaRbRcN⁺—, Q⁻ in which Ra, Rb and Rc, which may be identical or different, represent a hydrogen atom or a linear or branched C₁ to C₃₀ alkyl, preferably an alkyl, and Q⁻ represents an anionic counterion such as a halide, for instance a chloride or bromide;
   R′ represents an ammonium group R′aR′bR′cN⁺−, Q′⁻ in which R′a, R′b and R′c, which may be identical or different, represent a hydrogen atom or a linear or branched C₁ to C₃₀ alkyl, preferably an alkyl, and Q′⁻ represents an anionic counterion such as a halide, for instance a chloride or bromide;
it being understood that at least one of the radicals Ra, Rb, Rc, R′a, R′b and R′c represents a linear or branched C₈ to C₃₀ alkyl;
   n, x and y, which may be identical or different, represent an integer of between 1 and 10 000.

Preferably, in formula (Ib), at least one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radical(s) represent a linear or branched C₁-C₄ alkyl, notably methyl.

Preferably, in formula (Ib), only one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radicals represent a linear or branched C₁ to C₄ alkyl, notably methyl.

Better still, R may be a group chosen from —N+(CH₃)₃, Q′⁻ and —N⁺(C₁₂H₂₅)(CH₃)₂, Q′⁻, preferably a group —N⁺(CH₃)₃, Q′⁻.

Even better still, R′ may be a group —N⁺(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200®, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (Cis alkyl), such as the product Crodacel QS®, sold by the company Croda.

Mention may also be made of the hydroxyethylcelluloses of formula (Ib) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from the company Amerchol/Dow Chemical.

More particularly, the polymers of formula (Ib) are, for example, those whose viscosity is between 2000 and 3000 cPs inclusive, preferentially between 2700 and 2800 cPs. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs. Use may also be made of Softcat Polymer SX-1300X with a viscosity of between 1000 and 2000 cPs.

The cationic galactomannan gums are described more particularly in U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17, Jaguar C162 or Jaguar Excel by the company Rhodia. Such compounds have the INCI name guar hydroxypropyltrimonium chloride or hydroxypropyl guar hydroxypropyltrimonium chloride.
(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.
(4) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they include one or more tertiary amine functions, they can be quaternized;
(5) polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or under the name PD 170 or Delsette 101 by the company Hercules in the case of the adipic acid/epoxypropyl/diethylenetriamine copolymer.
(7) cyclopolymers of alkyldiallylamine or of dialkyldiallylammonium, such as homopolymers or copolymers including, as main constituent of the chain, units corresponding to formula (VI) or (VII):

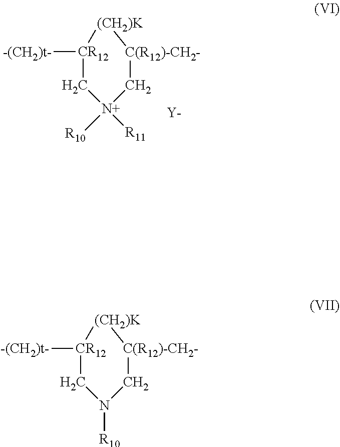

in which formulae (VI) and (VII):
   k and t are equal to 0 or 1, the sum k+t being equal to 1;
   R₁₂ denotes a hydrogen atom or a methyl radical;
   R₁₀ and R₁₁, independently of each other, denote an alkyl group containing from 1 to 6 carbon atoms, a hydroxyalkyl group in which the alkyl group contains 1 to 5 carbon atoms, a C₁ to C₄ amidoalkyl group; or alternatively R₁₀ and R₁₁ may denote, together with the nitrogen atom to which they are attached, heterocyclic groups such as piperidinyl or morpholinyl; R₁₀ and R₁₁, independently of each other, preferably denote an alkyl group containing from 1 to 4 carbon atoms; and
   Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold, for example, under the name Merquat 100 by the company Nalco (and homologues thereof of low weight-average molar masses) and the copolymers of diallyldimethylammonium salts (for example chloride) and of acrylamide, notably sold under the names Merquat 550 and Merquat 7SPR.
(8) quaternary diammonium polymers comprising repeating units of formula:

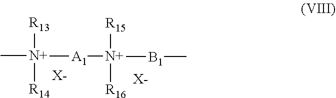

in which formula (VIII):
   R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second non-nitrogen heteroatom, or alternatively R₁₃, R₁₄, R₁₅ and R₁₆ represent a linear or branched C₁ to C₆ alkyl radical substituted with a nitrile, ester, acyl or amide group or a group —CO—O—R₁₇-D or —CO—NH—R₁₇-D where R₁₇ is an alkylene and D is a quaternary ammonium group;
   A₁ and B1 represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
   X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B1 can also denote a group (CH₂)ₙCO-D-OC—(CH₂)ₙ— in which D denotes:
   a) a glycol residue of formula —O—Z—O—, in which Z denotes a linear or branched hydrocarbon-based radical or a group corresponding to one of the following formulae: —(CH₂—CH₂-0)ₓ—CH₂—CH₂— and —[CH₂CH(CH₃)—O]_{y}—CH₂—CH(CH₃)—, where x and y denote an integer from 1 to 4, representing a defined and unique degree of polymerization or any number from 1 to 4 representing an average degree of polymerization;
   b) a bis-secondary diamine residue, such as a piperazine derivative;
   c) a bis-primary diamine residue of formula: —NH—Y—NH—, where Y denotes a linear or branched hydrocarbon-based radical, or alternatively the divalent radical —CH₂—CH₂—S—S—CH₂—CH₂—; or
   d) a ureylene group of formula: —NH—CO—NH—.

Preferably, X⁻ is an anion, such as chloride or bromide. These polymers have a number-average molar mass (Mn) generally of between 1000 and 100 000.

Mention may be made more particularly of polymers consisting of repeating units corresponding to the formula:

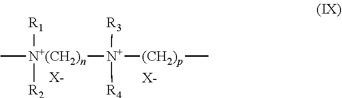

in which formula (IX) R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (IX) that is particularly preferred is the one for which R₁, R₂, R₃ and R₄ represent a methyl radical and n=3, p=6 and X=C₁, which is known as Hexadimethrine chloride according to the INCI (CTFA) nomenclature.
(9) polyquaternary ammonium polymers comprising units of formula (X):

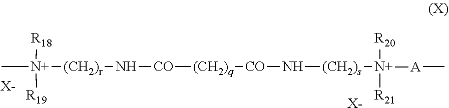

in which formula (X):
   R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
   r and s, which may be identical or different, are integers between 1 and 6,
   q is equal to 0 or to an integer between 1 and 34,
   X⁻ denotes an anion, such as a halide, and
   A denotes a dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by the company Miranol.
(10) quaternary polymers of vinylpyrrolidone and of vinylimidazole, for instance the products sold under the names Luviquat® FC 905, FC 550 and FC 370 by the company BASF.
(11) polyamines such as Polyquart® H sold by Cognis, which is referenced under the name Polyethylene Glycol (15) Tallow Polyamine in the CTFA dictionary.
(12) polymers including in their structure:
(a) one or more units corresponding to formula (A) below:

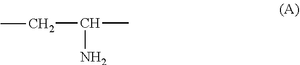

(b) optionally one or more units corresponding to formula (B) below:

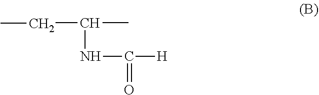

In other words, these polymers may be notably chosen from homopolymers or copolymers including one or more units derived from vinylamine and optionally one or more units derived from vinylformamide.
(13) and mixtures thereof.

Preferably, the cationic polymer(s) are chosen from cationic polysaccharides.

More preferentially, the cationic polymer(s) are chosen from cellulose ether derivatives including quaternary ammonium groups, cationic cellulose copolymers, cellulose derivatives grafted with a water-soluble quaternary ammonium monomer and cationic galactomannan gums, and mixtures thereof.

Even more preferentially, the cationic polymer(s) are chosen from cationic galactomannan gums, better still guar gums comprising cationic trialkylammonium groups, even better still guar gums modified with a 2,3-epoxypropyltrimethylammonium salt (for example a chloride).

Advantageously, when the cationic polymer(s) are present, the total content of cationic polymer(s) ranges from 0.1% to 15% by weight, more preferentially from 0.1% to 10% by weight, even more preferentially from 0.1% to 5% by weight, better still from 0.1% to 2% by weight, relative to the total weight of the composition.

Advantageously, when cationic galactomannan gums are present, the total content of cationic galactomannan gum(s) ranges from 0.1% to 15% by weight, more preferentially from 0.1% to 10% by weight, even more preferentially from 0.1% to 5% by weight, better still from 0.1% to 2% by weight, relative to the total weight of the composition.

### Additives

The composition according to the invention may contain any adjuvant or additive usually used.

Among the additives that may be used, mention may be made of reducing agents, thickeners, softeners, moisturizers, UV-screening agents, peptizers, solubilizers, fragrances, proteins, vitamins, polymers other than the polymers described previously, preserving agents, oils, waxes and mixtures thereof.

A subject of the present invention is also a cosmetic treatment process, and notably a process for washing keratin fibres, in particular human keratin fibres such as the hair, comprising the application to said keratin fibres of the composition according to the invention.

A subject of the present invention is also the use of the composition according to the invention for washing keratin fibres, in particular human keratin fibres such as the hair.

The present invention will now be described more specifically by means of examples, which do not in any way limit the scope of the invention. However, the examples make it possible to support specific characteristics, variants and preferred embodiments of the invention.

### EXAMPLES

In the examples that follow, all the amounts are given, unless otherwise indicated, as mass percentages of active material relative to the total weight of the composition.

### Example 1

450 g of fluid amino silicone (copolymer of dimethylsiloxane-aminoethylaminopropylmethylsiloxane bearing trimethylsilyl end groups, having an amine number of 7.2 mg of KOH/g and a viscosity of 5600 mPa·s at 25° C.) are transferred into a first vessel; 1800 g of dimethylsiloxane bearing trimethylsilyl end groups, having a viscosity of 61 500 mPa·s at 25° C., are added, with stirring, and the stirring is maintained for 2 hours at room temperature.

In a separate vessel, 49 g of steareth-6 and 62 g of PEG100 stearate are mixed, and the mixture is heated to 60° C. The mixture is maintained at this temperature until a liquid mixture is obtained, and 31 g of trideceth-3 and 350 g of trideceth-10 (80% of active material) are then added. The surfactant mixture has an HLB=11.25. 80 g of water and 6.2 g of glacial acetic acid are added and the stirring is continued until a creamy paste is obtained.

The content of this second vessel (creamy paste) is then transferred into the first vessel (containing the silicones), and the mixture obtained is then mixed for 30 minutes at room temperature (20-25° C.). The mixing steps are performed so as to obtain a homogeneous mixture; they are performed at room temperature.
79.6 g of demineralized water are added and mixing is performed for 60 minutes.
72.7 g of demineralized water are added and mixing is performed for 50 minutes.
197.4 g of demineralized water are added and mixing is performed for 5 minutes.
294.3 g of demineralized water are added and mixing is performed for 5 minutes.
180 g of demineralized water are added and mixing is performed for 5 minutes.
180 g of demineralized water are added and mixing is performed for 5 minutes.
197.4 g of demineralized water are added and mixing is performed for 5 minutes.
197.4 g of demineralized water are added and mixing is performed for 3 minutes.
228.5 g of demineralized water are added and mixing is performed for 3 minutes.

Finally, 40.5 g of 2-phenoxyethanol (preserving agent) are added and mixing is performed for 3 minutes.

An oil-in-water emulsion having a particle size D₅₀ of 170 nm is obtained.

### Example 2

Compositions A1, A2, A3 and A4 according to the invention, as described in Table 1 below, were prepared. The amounts are expressed as percentage of active material (% AM), unless otherwise mentioned.

TABLE 1

| | Compositions (% AM) | | | |
|---|---|---|---|---|
| | A1 | A2 | A3 | A4 |
| Sodium lauryl ether | 15.4 | 13.9 | 13.9 | 13.9 |
| sulfate | | | | |
| Cocamidopropyl betaine | 1.2 | 1.2 | 1.2 | 1.2 |
| Cocoyl betaine | 0.3 | 0.3 | 0.3 | 0.3 |
| PPG-5-Ceteth-20 | 0.6 | 0.6 | 0.6 | 0.6 |
| Carbomer | 0.2 | 0.2 | 0.2 | 0.2 |
| Guar | 0.25 | 0.25 | 0.25 | 0.25 |
| Hydroxypropyltrimonium | | | | |
| chloride | | | | |
| Dimethicone (and) | 4% emulsion | 3.6% emulsion | 3.6% emulsion | 3.6% emulsion |
| Amodimethicone (and) | (0.4% amodimethicone + | (0.36% amodimethicone + | (0.36% amodimethicone + | (0.36% amodimethicone + |
| Trideceth-10 (and) PEG- | 1.6% dimethicone) | 1.44% dimethicone) | 1.44% dimethicone) | 1.44% dimethicone) |
| 100 stearate (and) | | | | |
| Steareth-6 (and) | | | | |
| Trideceth-3 | | | | |
| Sodium chloride | 0.9 | 0.9 | 0.9 | 0.9 |
| Preserving agents | qs | qs | qs | qs |
| Glycol distearate | 1.6 | 1.6 | 1.6 | 1.6 |
| Glycerol | — | 2 | 2 | 2 |
| Lauric acid | 0.7 | — | — | 0.3 |
| Lauryl alcohol | — | 0.5 | — | — |
| Cetearyl alcohol | — | — | 0.5 | — |
| Stearamidopropyl- | — | — | — | 0.5 |
| dimethylamine | | | | |
| Water | qs 100 | qs 100 | qs 100 | qs 100 |

Compositions A1 to A4 according to the invention have good foam properties in terms of creaminess, smoothness and density of the foam. A rich, creamy foam is obtained.

The foam is easily spread over the entire head of hair and is stable: it holds well on the hair until rinsing is performed.

After rinsing and drying, the hair is smooth, shiny and easy to shape.

Compositions A5 to A7 according to the invention, as described in Table 2 below, were prepared. The amounts are expressed as percentage of active material (% AM), unless otherwise mentioned.

TABLE 2

| | Compositions (% AM) | | |
|---|---|---|---|
| | A5 | A6 | A7 |
| Sodium lauryl ether sulfate | 13.9 | 13.9 | 13.9 |
| Cocamidopropyl betaine | 1.2 | 1.2 | 1.2 |
| Cocoyl betaine | 0.3 | 0.3 | 0.3 |
| PPG-5-Ceteth-20 | 0.6 | 0.6 | 0.6 |
| Carbomer | 0.2 | 0.2 | 0.2 |
| Guar Hydroxypropyltrimonium | 0.25 | 0.25 | 0.25 |
| chloride | | | |
| Dimethicone (and) | 3.6% emulsion | 3.6% emulsion | 3.6% emulsion |
| Amodimethicone (and) | (0.36% amodimethicone + | (0.36% amodimethicone + | (0.36% amodimethicone + |
| Trideceth-10 (and) PEG-100 | 1.44% dimethicone) | 1.44% dimethicone) | 1.44% dimethicone) |
| stearate (and) Steareth-6 (and) | | | |
| Trideceth-3 | | | |
| Sodium chloride | 0.9 | 0.9 | 0.9 |
| Preserving agents | qs | qs | qs |
| Glycol distearate | 1.6 | 1.6 | 1.6 |
| Glycerol | 2 | 2 | 2 |
| Lauryl alcohol | — | 1 | — |
| Cetearyl alcohol | — | — | 1 |
| Stearamidopropyl- | 1 | — | — |
| dimethylamine | | | |
| Water | qs 100 | qs 100 | qs 100 |

Compositions A5 to A7 according to the invention have good foam properties in terms of creaminess, smoothness and density of the foam. The foam spreads easily over the entire head of hair.

After rinsing and drying, the hair is smooth, shiny and easy to shape.

### Characterization of Foam Density (Bubble Size)

The density of the foam of composition A4 was characterized by granulometry, i.e. by measuring the size of the air bubbles trapped in the foam by image analysis.

Composition A4 was diluted to 33% in demineralized water. The foam was then generated with an electric whisk and then placed in a quartz cuvette 100 μm deep.

Images were taken and image analysis performed with a 2× binocular magnifier and Granix® and Ellix® software. The results obtained are collated in Table 3 below:

TABLE 3

| | Invention |
|---|---|
| Mean bubble diameter (μm) | 162 ± 70 |
| Number of bubbles/mm² | 28 |

The composition according to the invention leads to a low mean diameter of the air bubbles and thus to a dense, smooth and creamy foam.

### Example 3

Composition A8 according to the invention and comparative composition B, as described in Table 4 below, were prepared. The amounts are expressed as percentage of active material (% AM), unless otherwise mentioned.

TABLE 4

| Compositions | A8 | B |
|---|---|---|
| Sodium lauryl ether sulfate | 15.4 | 15.4 |
| Cocamidopropyl betaine | 1.2 | 1.2 |
| Cocoyl betaine | 0.3 | 0.3 |
| PPG-5-Ceteth-20 | 0.6 | 0.6 |
| Carbomer | 0.2 | 0.2 |
| Guar Hydroxypropyltrimonium | 0.25 | 0.25 |
| chloride | | |
| Dimethicone (and) | 4% emulsion | 4% emulsion |
| Amodimethicone (and) | (0.4% amodimethicone + | (0.4% amodimethicone + |
| Trideceth-10 (and) PEG-100 | 1.6% dimethicone) | 1.6% dimethicone) |
| stearate (and) Steareth-6 (and) | | |
| Trideceth-3 | | |
| Lauric acid | 1 | — |
| Sodium chloride | 0.9 | 0.9 |
| Preserving agents | qs | qs |
| Glycol distearate | 1.6 | 1.6 |
| Water | qs 100 | qs 100 |

### Characterization of Foam Density (Bubble Size)

Each of compositions A8 and B was diluted to 33% in demineralized water. Then, 3 foam samples were produced for each of the compositions A8 and B, using an automated foam generator.

In order to observe the internal structure of the generated foams, a small amount of foam is crushed between two glass slides to trap only a single layer of bubble.

The thickness between the two glass slides is 100 μm. The bubble monolayer is observed with a Zeiss STEMI SV11 binocular loupe at ×2 magnification.

The estimation of the number of bubbles per mm² and the size distribution of the bubbles in 2 dimensions was carried out by digital image analysis (Grannix® and Ellix® software, three images analysed per sample). The results are in table 5 below.

The invention leads to smaller bubble sizes and also more bubbles per mm² giving a denser, creamier, smoother foam.

### Firmness of the Foam

Each of compositions A8 and B was diluted to 33% in demineralized water. Then, 2 foam samples were produced for each of the compositions A8 and B, using an automated foam generator.

The textural performance of the foam was evaluated by measuring its firmness, using a penetrometry protocol, performed on a TA.XT Plus texturometer.

In order to determine the firmness of the foams, the mobile of the texturometer penetrates the foam vertically over a distance of 40 mm and the resistance force (expressed in grams) of the foam on the mobile is measured.

The graph of the temporal evolution of the force measured during the penetration phase makes it possible to extract the parameters of interest: the firmness corresponds to the maximum value measured (average value of two tests).

The probe used is a polycarbonate disc of 5 mm thickness and 45 mm diameter.
Test speed: 40 mm/s
Penetration distance: 40 mm

The results are in table 6 below.

TABLE 6

| | Invention (A8) | Comparative (B) |
|---|---|---|
| Firmness (g) | 126.8 | 108.1 |

The force required to sink into foam A8 (according to the invention) is higher than that of foam B (comparative).

The foam generated by composition A8 (according to the invention) is firmer, denser and smoother than that generated by the comparative composition B.

### Cosmetic Performance: Suppleness and Lightness

Compositions A8 and B were applied to slightly sensitised hair strands (alkali solubility=20%, AS 20) at a rate of 0.4 g of composition per gram of hair.

The hair was then rinsed in a standardised manner, wrung out and dried in an oven.

The impact of the invention on performance in terms of lightness and suppleness was assessed on dry hair by 5 experts in a blind test on a rating scale from 0 (poor) to 5 (very good).

The assessment of suppleness is tactile. The expert takes hair in his hands and tries to bend it. He or she assesses whether the hair can be bent easily, whether it is malleable.

To assess lightness, the expert lifts the hair with his or her hands and looks at the way the hair falls: light hair is individual, flowing and does not fall in clumps.

The results obtained are collated in Table 7 below:

| | Suppleness | | Lightness | |
|---|---|---|---|---|
| | A8 | B | A8 | B |
| | (invention) | (comparative) | (invention) | (comparative) |
| Expert 1 | 3 | 2 | 3.5 | 2.5 |
| Expert 2 | 2.5 | 1.5 | 2.5 | 2 |
| Expert 3 | 3 | 2 | 4 | 3 |
| Expert 4 | 3 | 2 | 3 | 2 |
| Expert 5 | 3.5 | 2.5 | 3.5 | 2 |
| Mean | 3 | 2 | 3.3 | 2.3 |
| Standard deviation | 0.4 | 0.4 | 0.6 | 0.4 |

It clearly appears that the suppleness and lightness provided by composition A8 according to the invention were improved compared to those obtained with the comparative composition B. These results are significant in view of standard deviations.

1-20. (canceled)

21. A cosmetic composition comprising:
a) at least one anionic surfactant;
b) at least one amphoteric or zwitterionic surfactant;
c) at least one oil-in-water emulsion having a particle size D50 of less than 350 nm on a volume basis, the at least one oil-in-water emulsion comprising:
a silicone mixture comprising (i) one or more polydialkylsiloxanes comprising trialkylsilyl end groups and having a viscosity at 25° C. ranging from 40,000 to 100,000 mPa·s, and (ii) one or more amino silicones having a viscosity at 25° C. ranging from 1,000 to 15,000 mPa·s and an amine number ranging from 2 to 10 mg of KOH per gram of amino silicone;
a surfactant mixture comprising one or more nonionic surfactants, wherein the surfactant mixture has an HLB ranging from 10 to 16; and
water; and
d) at least 0.01% by weight, relative to the total weight of the composition, of one or more agents chosen from fatty amines, fatty acids, fatty alcohols, or mixtures thereof.

22. The composition of claim 21, wherein the anionic surfactant(s) are chosen from sulfate-type surfactants.

23. The composition of claim 22, wherein the anionic surfactant(s) are chosen from alkyl sulfates, alkyl ether sulfates, alkylamido sulfates, alkylamido ether sulfates, alkylaryl polyether sulfates, monoglyceride sulfates, salts thereof, or mixtures thereof.

24. The composition of claim 21, wherein the anionic surfactant(s) are chosen from alkyl sulfates, alkyl ether sulfates, or mixtures thereof.

25. The composition of claim 21, wherein the total amount of anionic surfactant(s) ranges from 1% to 50% by weight, relative to the total weight of the composition.

26. The composition of claim 21, wherein the at least one amphoteric or zwitterionic surfactant is chosen from (C₈-C₂₀)alkylbetaines, (C₈-C₂₀)alkylamido(C₃-C₈)alkylbetaines, or mixtures thereof.

27. The composition of claim 21, wherein the total amount of the amphoteric or zwitterionic surfactant(s) ranges from 0.1% to 30% by weight, relative to the total weight of the composition.

28. The composition of claim 21, wherein the polydialkylsiloxane(s) having trialkylsilyl end groups are compounds of formula (I): 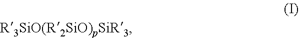
wherein:
R′ is independently chosen from a monovalent hydrocarbon-based radical comprising from 1 to 18 carbon atoms; and
p is an integer ranging from 500 to 2000.

29. The composition of claim 21, wherein the polydialkylsiloxanes having trialkylsilyl end groups have a viscosity ranging from 40,000 to 70,000 mPa·s at 25° C.

30. The composition of claim 21, wherein the amino silicone(s) are compounds of formula (II): XR₂Si(OSiAR)ₙ(OSiR₂)ₘOSiR₂X (II), wherein:
R is independently chosen from a monovalent hydrocarbon-based radical having from 1 to 18 carbon atoms;
X is independently chosen from R or a hydroxyl (OH) or a C₁-C₆ alkoxy group;
A is an amino radical of formula —R¹—[NR²—R³—]ₓNR²2, or the protonated form of formula —R¹—[NR²—R³—]ₓNR²₂, wherein:
R¹ represents a C₁-C₆ alkylene radical,
R² is independently chosen from a hydrogen atom or a C₁-C₄ alkyl radical,
R³ is a C₁-C₆ alkylene radical, and
x is an integer equal to 0 or 1;
m and n are integers ranging from 50 to 1000.

31. The composition of claim 21, wherein the silicone mixture comprises (i) polydialkylsiloxanes in a total amount of from 70% to 90%, and (ii) amino silicones in a total amount of from 10% to 30%, wherein all amounts are measured by weight, relative to the total weight of the silicone mixture.

32. The composition of claim 21, wherein the nonionic surfactants are chosen from:
(i) (poly)oxyalkylenated fatty alcohols of formula: R₃—(OCH₂CH₂)_{c}OH wherein:
R₃ represents a linear or branched alkyl or alkenyl radical comprising from 8 to 40 carbon atoms; and
c is an integer ranging from 1 to 200,
(ii) (poly)oxyalkylenated (C₈-C₃₂)alkyl phenyl ethers;
(iii) polyoxyalkylenated esters of C₈-C₃₂ fatty acids and of sorbitan; and
(iv) polyoxyethylenated esters of C₈-C₃₂ fatty acids.

33. The composition of claim 21, wherein the oil-in-water emulsion has a particle size D50 of between 100 and 300 nm.

34. The composition of claim 21, wherein the total amount of oil-in-water emulsion ranges from 0.1% to 10% by weight, relative to the total weight of the composition.

35. The composition of claim 21, wherein the fatty amines are chosen from fatty amidoamines.

36. The composition of claim 21, wherein the fatty acids are chosen from linear or branched, saturated or unsaturated fatty acids comprising from 8 to 30 carbon atoms, or mixtures thereof.

37. The composition of claim 21, wherein the fatty alcohols are chosen from saturated or unsaturated, linear or branched fatty alcohols comprising from 8 to 30 carbon atoms, or mixtures thereof.

38. The composition of claim 21, wherein the total amount of agents ranges from 0.01% to 5% by weight, relative to the total weight of the composition.

39. A method for washing keratin fibres, comprising applying to the keratin fibres a composition comprising:
a) at least one anionic surfactant;
b) at least one amphoteric or zwitterionic surfactant;
c) at least one oil-in-water emulsion having a particle size D50 of less than 350 nm on a volume basis, and comprising:
a silicone mixture comprising (i) one or more polydialkylsiloxanes comprising trialkylsilyl end groups and having a viscosity at 25° C. ranging from 40,000 to 100,000 mPa·s and (ii) one or more amino silicones having a viscosity at 25° C. ranging from 1,000 to 15,000 mPa·s and having an amine number ranging from 2 to 10 mg of KOH per gram of amino silicone;
a surfactant mixture comprising one or more nonionic surfactants, said mixture having an HLB ranging from 10 to 16; and
water; and
d) at least 0.01% by weight, relative to the total weight of the composition, of one or more agents chosen from fatty amines, fatty acids, fatty alcohols, or mixtures thereof.

40. A method comprising applying to human keratin fibres a composition comprising:
a) at least one anionic surfactant;
b) at least one amphoteric or zwitterionic surfactant;
c) at least one oil-in-water emulsion having a particle size D50 of less than 350 nm on a volume basis, and comprising:
a silicone mixture comprising (i) one or more polydialkylsiloxanes comprising trialkylsilyl end groups and having a viscosity at 25° C. ranging from 40 000 to 100 000 mPa·s and (ii) one or more amino silicones having a viscosity at 25° C. ranging from 1000 to 15 000 mPa·s and having an amine number ranging from 2 to 10 mg of KOH per gram of amino silicone;
a surfactant mixture comprising one or more nonionic surfactants, said mixture having an HLB ranging from 10 to 16; and
water; and
d) at least 0.01% by weight, relative to the total weight of the composition, of one or more agents chosen from fatty amines, fatty acids, fatty alcohols, or mixtures thereof.
